(19) United States
(12) Patent Application Publication
(10) Pub. No.: US 2024/0152808 A1
(43) Pub. Date: May 9, 2024

(54) METHOD, SYSTEM, AND NON-TRANSITORY COMPUTER-READABLE RECORDING MEDIUM FOR MANAGING TRAINING DATA OF BIOSIGNAL ANALYSIS MODEL

(71) Applicant: HUINNO, CO., LTD., Seoul (KR)

(72) Inventors: Jun Sang PARK, Goyang-si (KR); Jun Ho AN, Seoul (KR)

(21) Appl. No.: 18/412,935

(22) Filed: Jan. 15, 2024

Related U.S. Application Data
(63) Continuation of application No. PCT/KR2022/009927, filed on Jul. 8, 2022.

(30) Foreign Application Priority Data
Jul. 20, 2021 (KR) 10-2021-0095174

(51) Int. Cl. G06N 20/00

(52) U.S. Cl. CPC G06N 20/00

(57) ABSTRACT

A method for managing training data of a biosignal analysis model includes the steps of: converting data on at least one of a plurality of leads into augmented data associated with a specific lead among the plurality of leads; and training an analysis model for determining arrhythmia on the basis of data on the specific lead, using the augmented data and the data on the specific lead as training data.

### CROSS-REFERENCE TO RELATED APPLICATION(S)

This application is a Continuation of International Application No. PCT/KR2022/009927 filed on Jul. 8, 2022, which claims priority to Korean Patent Application No. 10-2021-0095174 filed on Jul. 20, 2021. The aforementioned applications are incorporated herein by reference in their entireties.

### TECHNICAL FIELD

The present invention relates to a method, system, and non-transitory computer-readable recording medium for managing training data of a biosignal analysis model.

### RELATED ART

Due to recent rapid progress in science and technology, the quality of life of all mankind is being enhanced and medical environment has changed a great deal. Particularly, in recent years, wearable monitoring devices that can analyze electrocardiograms (ECGs) and determine arrhythmia during daily life without visiting a hospital have become widely available to the public.

In general, a method of analyzing a 12-lead ECG to determine arrhythmia is widely used. Although it is necessary to form multiple contact points on a subject's body part in order to analyze the 12-lead ECG, wearable monitoring devices are typically limited in their ability to form multiple contact points on a subject's body part, so that only data on a specific lead among data on the 12-lead ECG is analyzed to determine arrhythmia.

These wearable monitoring devices are provided with an artificial intelligence model for analyzing data on a specific lead. Conventionally, only the data on the specific lead has been used as training data for training the artificial intelligence model.

However, when the artificial intelligence model is trained using only the data on the specific lead, it is possible to make precise determinations of some types of arrhythmia that can be accurately determined by analyzing only the data on the specific lead, whereas it is difficult to make precise determinations of types of arrhythmia that can be accurately determined only by comprehensively analyzing the data on the specific lead and data on other leads.

### SUMMARY

One object of the present invention is to solve all the above-described problems in the prior art.

Another object of the invention is to train an artificial intelligence model for determining arrhythmia on the basis of data on a specific lead, using data on multiple leads, so that the artificial intelligence model may determine various types of arrhythmia with high precision.

The representative configurations of the invention to achieve the above objects are described below.

According to one aspect of the invention, there is provided a method for managing training data of a biosignal analysis model, the method comprising the steps of: converting data on at least one of a plurality of leads into augmented data associated with a specific lead among the plurality of leads; and training an analysis model for determining arrhythmia on the basis of data on the specific lead, using the augmented data and the data on the specific lead as training data.

According to another aspect of the invention, there is provided a system for managing training data of a biosignal analysis model, the system comprising: a data management unit configured to convert data on at least one of a plurality of leads into augmented data associated with a specific lead among the plurality of leads; and a training management unit configured to train an analysis model for determining arrhythmia on the basis of data on the specific lead, using the augmented data and the data on the specific lead as training data.

In addition, there are further provided other methods and systems to implement the invention, as well as non-transitory computer-readable recording media having stored thereon computer programs for executing the methods.

According to the invention, it is possible to train an artificial intelligence model for determining arrhythmia on the basis of data on a specific lead, using data on multiple leads, so that the artificial intelligence model may determine various types of arrhythmia with high precision.

### BRIEF DESCRIPTION OF THE DRAWINGS

FIG. 1 schematically shows the configuration of an entire system for managing training data of a biosignal analysis model according to one embodiment of the invention

FIG. 2 specifically shows the internal configuration of a training data management system according to one embodiment of the invention.

FIG. 3 illustratively shows vectorized data on a plurality of leads according to one embodiment of the invention.

FIG. 4 illustratively shows vectorized data on other leads and vectorized data on a specific lead according to one embodiment of the invention.

FIG. 5 illustratively shows augmented data associated with a specific lead according to one embodiment of the invention.

### DETAILED DESCRIPTION

In the following detailed description of the present invention, references are made to the accompanying drawings that show, by way of illustration, specific embodiments in which the invention may be practiced. These embodiments are described in sufficient detail to enable those skilled in the art to practice the invention. It is to be understood that the various embodiments of the invention, although different from each other, are not necessarily mutually exclusive. For example, specific shapes, structures and characteristics described herein may be implemented as modified from one embodiment to another without departing from the spirit and scope of the invention. Furthermore, it shall be understood that the positions or arrangements of individual elements within each embodiment may also be modified without departing from the spirit and scope of the invention. Therefore, the following detailed description is not to be taken in a limiting sense, and the scope of the invention is to be taken as encompassing the scope of the appended claims and all equivalents thereof. In the drawings, like reference numerals refer to the same or similar elements throughout the several views.

Hereinafter, various preferred embodiments of the invention will be described in detail with reference to the accompanying drawings to enable those skilled in the art to easily implement the invention.

### Configuration of the Entire System

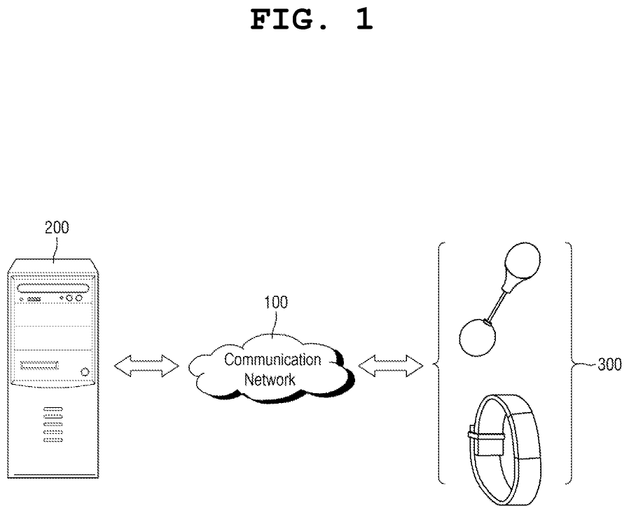

FIG. 1 schematically shows the configuration of the entire system for managing training data of a biosignal analysis model according to one embodiment of the invention

As shown in FIG. 1, the entire system according to one embodiment of the invention may comprise a communication network 100, a training data management system 200, and a device 300.

First, the communication network 100 according to one embodiment of the invention may be implemented regardless of communication modality such as wired and wireless communications, and may be constructed from a variety of communication networks such as local area networks (LANs), metropolitan area networks (MANs), and wide area networks (WANs). Preferably, the communication network 100 described herein may be the Internet or the World Wide Web (WWW). However, the communication network 100 is not necessarily limited thereto, and may at least partially include known wired/wireless data communication networks, known telephone networks, or known wired/wireless television communication networks.

For example, the communication network 100 may be a wireless data communication network, at least a part of which may be implemented with a conventional communication scheme such as radio frequency (RF) communication, WiFi communication, cellular communication (e.g., Long Term Evolution (LTE) communication), Bluetooth communication (more specifically, Bluetooth Low Energy (BLE) communication), infrared communication, and ultrasonic communication.

Next, the training data management system 200 according to one embodiment of the invention may function to convert data on at least one of a plurality of leads into augmented data associated with a specific lead among the plurality of leads, and train an analysis model for determining arrhythmia on the basis of data on the specific lead, using the augmented data and the data on the specific lead as training data.

The configuration and functions of the training data management system 200 according to one embodiment of the invention will be discussed in detail below.

Next, the device 300 according to one embodiment of the invention is digital equipment capable of connecting to and then communicating with the training data management system 200, and any type of digital equipment having a memory means and a microprocessor for computing capabilities, such as a smart phone, a tablet, a smart watch, a smart patch, a smart band, smart glasses, a desktop computer, a notebook computer, a workstation, a personal digital assistant (PDAs), a web pad, and a mobile phone, may be adopted as the device 300 according to the invention.

In particular, the device 300 according to one embodiment of the invention may include a sensing means (e.g., a contact electrode) for acquiring a biosignal (e.g., an ECG) from a human body.

Meanwhile, the device 300 may include an application (not shown) for assisting a user to receive services according to the invention from the training data management system 200. The application may be downloaded from the training data management system 200 or an external application distribution server (not shown). Meanwhile, the characteristics of the application may be generally similar to those of a data management unit 210, a training management unit 220, a communication unit 230, and a control unit 240 of the training data management system 200 to be described below. Here, at least a part of the application may be replaced with a hardware device or a firmware device that may perform a substantially equal or equivalent function, as necessary.

### Configuration of the Training Data Management System

Hereinafter, the internal configuration of the training data management system 200 crucial for implementing the invention and the functions of the respective components thereof will be discussed.

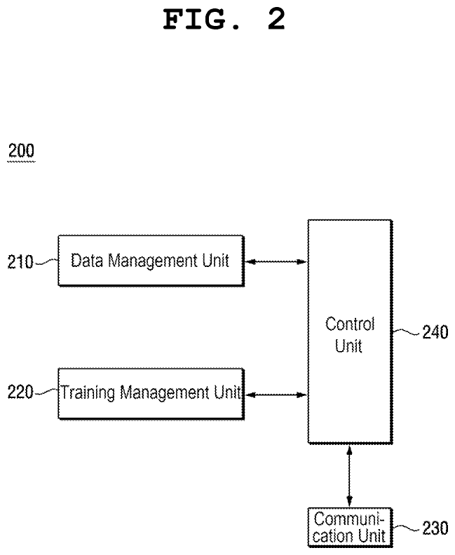

FIG. 2 specifically shows the internal configuration of the training data management system 200 according to one embodiment of the invention.

As shown in FIG. 2, the training data management system 200 according to one embodiment of the invention may comprise a data management unit 210, a training management unit 220, a communication unit 230, and a control unit 240. According to one embodiment of the invention, at least some of the data management unit 210, the training management unit 220, the communication unit 230, and the control unit 240 of the training data management system 200 may be program modules to communicate with an external system (not shown). The program modules may be included in the training data management system 200 in the form of operating systems, application program modules, or other program modules, while they may be physically stored in a variety of commonly known storage devices. Further, the program modules may also be stored in a remote storage device that may communicate with the training data management system 200. Meanwhile, such program modules may include, but are not limited to, routines, subroutines, programs, objects, components, data structures, and the like for performing specific tasks or executing specific abstract data types as will be described below in accordance with the invention.

Meanwhile, the above description is illustrative although the training data management system 200 has been described as above, and it will be apparent to those skilled in the art that at least a part of the components or functions of the training data management system 200 may be implemented in the device 300 or a server (not shown) or included in an external system (not shown), as necessary.

First, the data management unit 210 according to one embodiment of the invention may function to convert data on at least one of a plurality of leads into augmented data associated with a specific lead among the plurality of leads.

The data management unit 210 according to one embodiment of the invention may acquire data on the plurality of leads from at least one electrocardiograph (e.g., a 12-lead electrocardiograph, a Holter electrocardiograph, or a wearable electrocardiograph). Here, the data on the plurality of leads may be data on a 12-lead ECG, and may include data on lead I, lead II, lead III, lead aVR, lead aVL, lead aVF, lead V1, lead V2, lead V3, lead V4, lead V5, and lead V6.

Next, the data management unit 210 according to one embodiment of the invention may extract data on a specific lead and data on at least one other lead other than the data on the specific lead (hereinafter referred to as “data on other leads”) from the data on the plurality of leads acquired from the at least one electrocardiograph. The data on the specific lead may be training data used to train an analysis model for determining arrhythmia, and may be the same type of data (i.e., data measured on the same lead) as test input data that is inputted to the analysis model during an analysis process. Further, the data on other leads may be training data that may be further used together with the data on the specific lead to train the analysis model for determining arrhythmia, and may be data measured on other leads having a predetermined relationship with the data on the specific lead.

Specifically, the data management unit 210 according to one embodiment of the invention may organize, into a database, combinations of data to be used as the training data of the analysis model for each test input data inputted to the analysis model, and may extract the data on the specific lead and the data on other leads from the data on the plurality of leads acquired from the at least one electrocardiograph on the basis of the combinations. For example, when the test input data inputted to the analysis model is the data on lead II, the data management unit 210 according to one embodiment of the invention may extract the data on lead II as the data on the specific lead, and extract the data on lead aVR and the data on lead I as the data on other leads, from the data on the plurality of leads acquired from the at least one electrocardiograph, with reference to one of the above combinations.

Meanwhile, the data management unit 210 according to one embodiment of the invention may convert the extracted data on other leads into augmented data associated with the specific lead. For example, the data management unit 210 according to one embodiment of the invention may convert the data on lead aVR and the data on lead I extracted as the data on other leads into augmented data associated with the data on lead II extracted as the data on the specific lead.

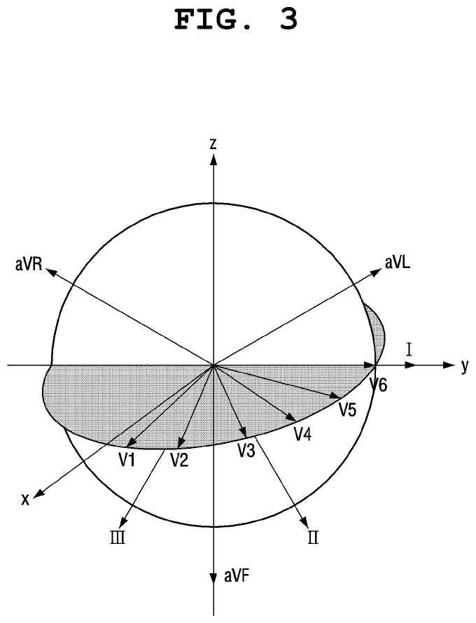

Specifically, the data management unit 210 according to one embodiment of the invention may vectorize the data on other leads and the data on the specific lead. More specifically, the data management unit 210 according to one embodiment of the invention may vectorize the data on other leads and the data on the specific lead in a three-dimensional space. Referring to FIG. 3, the data on lead I, lead II, lead III, lead aVL, lead aVR, and lead aVF among the data on the 12-lead ECG may be vectorized with respect to a y-z plane of the three-dimensional space. Further, the data on lead V1, lead V2, lead V3, lead V4, lead V5, and lead V6 among the data on the 12-lead ECG may be vectorized with respect to a x-y plane of the three-dimensional space. Here, the y-z plane of the three-dimensional space may be associated with a longitudinal section of a human body, and the x-y plane of the three-dimensional space may be associated with a transverse section of the human body.

Next, the data management unit 210 according to one embodiment of the invention may calculate information on a difference between the vectorized data on other leads and the vectorized data on the specific lead. Specifically, the data management unit 210 according to one embodiment of the invention may calculate at least one of information on a phase difference and information on a magnitude difference as the information on the difference between the vectorized data on other leads and the vectorized data on the specific lead.

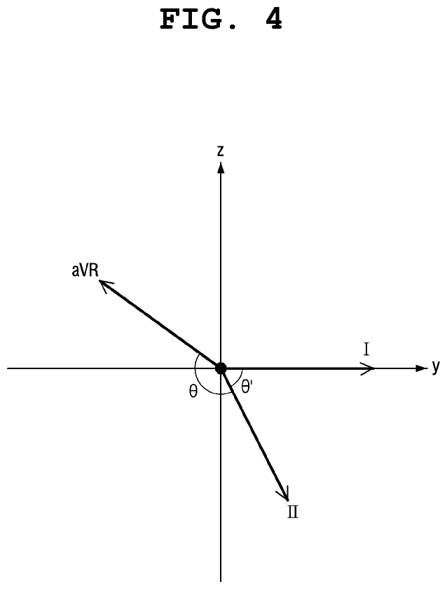

For example, referring to FIG. 4, the data management unit 210 according to one embodiment of the invention may vectorize the data on lead aVR and the data on lead I as the data on other leads, and vectorize the data on lead II as the data on the specific lead, with respect to the y-z plane. The data management unit 210 according to one embodiment of the invention may calculate θ as information on a phase difference between the vectorized data on lead aVR and the vectorized data on lead II, and may calculate θ′ as information on a phase difference between the vectorized data on lead I and the vectorized data on lead II. Meanwhile, although not shown in FIG. 4, the data management unit 210 according to one embodiment of the invention may also calculate information on a magnitude difference between the vectorized data on lead aVR (or the vectorized data on lead I) and the vectorized data on lead II when the magnitude difference exists between the vectorized data on lead aVR (or the vectorized data on lead I) and the vectorized data on lead II.

Next, the data management unit 210 according to one embodiment of the invention may convert the data on other leads into augmented data associated with the specific lead by correcting the data on other leads with respect to the data on the specific lead with reference to the calculated information on the difference.

Specifically, when the difference between the vectorized data on other leads and the vectorized data on the specific lead is not less than a predetermined level, the data management unit 210 according to one embodiment of the invention may convert the data on other leads into the augmented data associated with the specific lead by correcting the data on other leads with respect to the data on the specific lead. More specifically, when the phase difference between the vectorized data on other leads and the vectorized data on the specific lead is not less than a predetermined level, the data management unit 210 according to one embodiment of the invention may convert the data on other leads into the augmented data associated with the specific lead by correcting the phase of the data on other leads with respect to the phase of the data on the specific lead. Further, when the magnitude difference between the vectorized data on other leads and the vectorized data on the specific lead is not less than a predetermined level, the data management unit 210 according to one embodiment of the invention may convert the data on other leads into the augmented data associated with the specific lead by correcting the magnitude of the data on other leads with respect to the magnitude of the data on the specific lead.

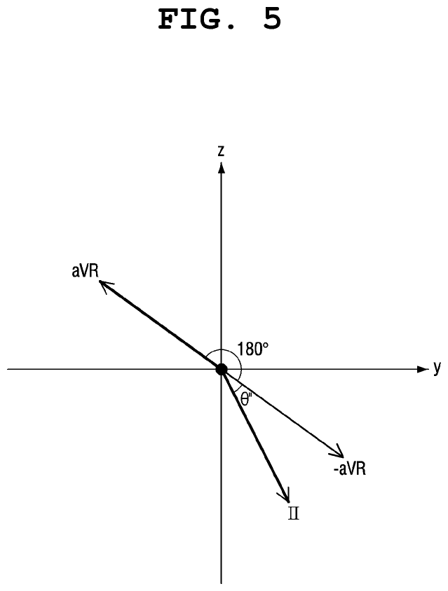

For example, referring to FIG. 5, the data management unit 210 according to one embodiment of the invention may convert the data on lead aVR into augmented data associated with the data on lead II by determining that a phase difference between the vectorized data on lead aVR and the vectorized data on lead II is not less than a predetermined level (e.g., 90°), and correcting the phase of the data on lead aVR with respect to the phase of the data on lead II (e.g., correcting the phase of the data on lead aVR by 180°). Here, the augmented data associated with the data on lead II may be data on lead −aVR, and a phase difference θ″ between the data on lead II and the data on lead −aVR may be less than a predetermined level (e.g., 90°).

Meanwhile, when the difference between the vectorized data on other leads and the vectorized data on the specific lead is less than a predetermined level, the data management unit 210 according to one embodiment of the invention may not correct the data on other leads. In this case, the augmented data associated with the specific lead may include the uncorrected data on other leads.

For example, referring again to FIG. 4, the data management unit 210 according to one embodiment of the invention may determine that the phase difference θ′ between the vectorized data on lead I and the vectorized data on lead II is less than a predetermined level (e.g., 90°), and may not correct the phase of the data on lead I with respect to the phase of the data on lead II. Here, the augmented data associated with the data on lead II may be the uncorrected data on lead I itself.

However, the manner in which the data management unit 210 converts the data on other leads into the augmented data associated with the specific lead according to one embodiment of the invention is not necessarily limited to the above examples, and may be diversely changed as long as the objects of the invention may be achieved.

Next, the training management unit 220 according to one embodiment of the invention may function to train an analysis model for determining arrhythmia on the basis of data on the specific lead, using the augmented data associated with the specific lead and the data on the specific lead as training data.

Specifically, according to one embodiment of the invention, the analysis model trained by the training management unit 220 may analyze the data on the specific lead measured from a subject to determine arrhythmia of the subject. The training management unit 220 according to one embodiment of the invention may train the analysis model using not only the data on the specific lead but also the augmented data associated with the specific lead as training data. That is, according to one embodiment of the invention, the analysis model may be trained on the basis of data on multiple leads (i.e., the data on the specific lead and the augmented data associated with the specific lead), so that various types of arrhythmia may be determined with high precision even in an environment where only data on a single lead (i.e., the data on the specific lead) is inputted as test input data.

Next, the communication unit 230 according to one embodiment of the invention may function to enable data transmission/reception from/to the data management unit 210 and the training management unit 220.

Lastly, the control unit 240 according to one embodiment of the invention may function to control data flow among the data management unit 210, the training management unit 220, and the communication unit 230. That is, the control unit 240 according to the invention may control data flow into/out of the training data management system 200 or data flow among the respective components of the training data management system 200, such that the data management unit 210, the training management unit 220, and the communication unit 230 may carry out their particular functions, respectively.

The embodiments according to the invention as described above may be implemented in the form of program instructions that can be executed by various computer components, and may be stored on a computer-readable recording medium. The computer-readable recording medium may include program instructions, data files, and data structures, separately or in combination. The program instructions stored on the computer-readable recording medium may be specially designed and configured for the present invention, or may also be known and available to those skilled in the computer software field. Examples of the computer-readable recording medium include the following: magnetic media such as hard disks, floppy disks and magnetic tapes; optical media such as compact disk-read only memory (CD-ROM) and digital versatile disks (DVDs); magneto-optical media such as floptical disks; and hardware devices such as read-only memory (ROM), random access memory (RAM) and flash memory, which are specially configured to store and execute program instructions. Examples of the program instructions include not only machine language codes created by a compiler, but also high-level language codes that can be executed by a computer using an interpreter. The above hardware devices may be changed to one or more software modules to perform the processes of the present invention, and vice versa.

Although the present invention has been described above in terms of specific items such as detailed elements as well as the limited embodiments and the drawings, they are only provided to help more general understanding of the invention, and the present invention is not limited to the above embodiments. It will be appreciated by those skilled in the art to which the present invention pertains that various modifications and changes may be made from the above description.

Therefore, the spirit of the present invention shall not be limited to the above-described embodiments, and the entire scope of the appended claims and their equivalents will fall within the scope and spirit of the invention.

What is claimed is:

1. A method for managing training data of a biosignal analysis model, the method comprising steps of:
converting data on at least one of a plurality of leads into augmented data associated with a specific lead among the plurality of leads; and
training an analysis model for determining arrhythmia on the basis of data on the specific lead, using the augmented data and the data on the specific lead as training data.

2. The method of claim 1, wherein in the converting step, the data on the at least one lead and the data on the specific lead are vectorized.

3. The method of claim 2, wherein in the converting step, information on a difference between the vectorized data on the at least one lead and the vectorized data on the specific lead is calculated.

4. The method of claim 3, wherein in the converting step, the data on the at least one lead is converted into the augmented data by correcting the data on the at least one lead with respect to the data on the specific lead with reference to the calculated information on the difference.

5. The method of claim 4, wherein the calculated information on the difference includes at least one of information on a phase difference and information on a magnitude difference.

6. A non-transitory computer-readable recording medium having stored thereon a computer program for executing the method of claim 1.

7. A system for managing training data of a biosignal analysis model, the system comprising:
a data management unit configured to convert data on at least one of a plurality of leads into augmented data associated with a specific lead among the plurality of leads; and
a training management unit configured to train an analysis model for determining arrhythmia on the basis of data on the specific lead, using the augmented data and the data on the specific lead as training data.

8. The system of claim 7, wherein the data management unit is configured to vectorize the data on the at least one lead and the data on the specific lead.

9. The system of claim 8, wherein the data management unit is configured to calculate information on a difference between the vectorized data on the at least one lead and the vectorized data on the specific lead.

10. The system of claim 9, wherein the data management unit is configured to convert the data on the at least one lead into the augmented data by correcting the data on the at least one lead with respect to the data on the specific lead with reference to the calculated information on the difference.

11. The system of claim 10, wherein the calculated information on the difference includes at least one of information on a phase difference and information on a magnitude difference.
